(19) United States
(12) Patent Application Publication
(10) Pub. No.: US 2024/0366491 A1
(43) Pub. Date: Nov. 7, 2024

(54) COSMETIC COMPOSITION FOR THE HAIR AND USE THEREOF FOR HAIR AND/OR SCALP CARE

(71) Applicant: PIERRE FABRE DERMO-COSMETIQUE, LAVAUR (FR)

(72) Inventor: Sabine BIAIS, CASTRES (FR)

(21) Appl. No.: 18/573,044

(22) PCT Filed: Jun. 24, 2022
(86) PCT No.: PCT/EP2022/067305

§ 371 Date: Dec. 21, 2023

(30) Foreign Application Priority Data
Jun. 29, 2021 (FR) 2106979

(51) Int. Cl. A61K 8/73; A61K 8/42; A61Q 5/12

(52) U.S. Cl. CPC A61K 8/732; A61K 8/42; A61Q 5/12; A61K 2800/34

(57) ABSTRACT

A cosmetic composition for the hair suitable for topical application, including a mixture of at least one microbial polysaccharide, at least one modified starch and at least one cationic surfactant, in a cosmetically acceptable carrier, the composition being free of silicones and quaternary ammonium. The composition is particularly suitable for use as a conditioner for hair and/or scalp care.

The present invention relates to a new hair-type cosmetic composition, i.e. intended for the care of the hair and/or the scalp. The invention also relates to the use of such a composition in cosmetics, and in particular the use of such a composition as a conditioner for the care of the scalp and/or the hair.

For example, the hair care compositions may consist of conditioning shampoos, conditioners, masks or sera. They may be in the form of gels, hair lotions, or more or less thick care creams.

Many hair care compositions proposed by the prior art comprise silicones, which are used therein as conditioning agents. In particular, silicones ensure a slippery effect promoting a good distribution of the composition over the application area, in particular over the length of the hair. Nonetheless, silicones have the drawback of strongly loading and weighting the hair, which leads to the phenomenon commonly called “build-up effect”, i.e. a hair which quickly becomes greasy again and a feeling of poorly rinsed locks. Furthermore, it has been discovered in the last few years that silicones have the drawback of choking the hair, and consequently damaging them, so that their use within hair cosmetic compositions has become undesirable. Moreover, silicones are also criticised for their environmental impact.

Hence, it has seemed desirable to provide compositions for hair care, in particular of the conditioner type, which are free of silicones. In the present description, by conditioner, it is conventionally meant a cosmetic product intended to untangle, embellish and cure the hair, the conditioner being generally applied after the shampoo.

Hence, a current concern in the field of hair care, in particular of conditioning care, is to provide compositions that are free of silicones, but which, upon application, have a slippery effect similar to that obtained with the silicones, so that the user has the feeling that the distribution of the composition is sufficient over the entirety of his/her hair, that being so while conferring good cosmetic properties on the hair, such as smoothing, ease of untangling and shine, and without the user having the feeling that his/her hair is heavy and that it easily become greasy again, in particular if his/her hair is fine.

Cosmetic compositions for the care of the skin or the hair, free of silicone, based, among other ingredients, on xanthan gum, starch or modified starch and cationic surfactant, have been proposed by the prior art.

The document WO 2015/075236 describes a cosmetic composition for caring for and/or conditioning keratin fibres, comprising a silane, one or more anionic and/or non-ionic polysaccharide(s) and at least one water-soluble mineral salt. However, none of these prior art compositions is satisfactory for the care of both the hair and the scalp, in particular in terms of ease of distribution of the composition both on the hair and on the scalp and of impregnation capacity. The present invention aims to provide a composition which meets all of these properties, i.e. which allows obtaining all of the expected benefits of a conditioner, by having, despite the absence of silicones, a good ease of distribution and a good capacity of impregnation of the scalp but also of the entire hair, all that without rendering the latter heavy. The invention also aims that such properties are obtained without implementing products likely to harm the environment, in particular without implementing quaternary ammoniums, surfactant substances used in many hair cosmetic compositions proposed by the prior art.

Additional objectives of the invention are that the hair cosmetic composition it proposes is easy to dose by the user, so as to avoid an overconsumption thereof at each application, and that it is beneficial both for the hair and for the scalp, unlike the hair cosmetic compositions proposed by the prior art, which are generally suitable for the care of only one or the other.

The applicant has now discovered that, surprisingly, compositions comprising a combination of two particular ingredients with gelling properties and of a specific ingredient with conditioner properties, more particularly, at least one microbial polysaccharide, at least one modified starch and at least one conditioning agent selected from among cationic surfactants, allowed achieving the above-mentioned objectives, and in particular were both beneficial for the scalp and untangling for the hair, while they were free of silicones and quaternary ammoniums.

In particular, the applicant has noticed that it was not necessary to use this composition in large amounts, and that during the application to the hair, it could be distributed and spread easily from the roots to the tips, the rinsing being particularly easy.

It has also noticed that this composition was perfectly suitable for the scalp but also for the hair, in particular thanks to its gelled texture, which enables easy distribution thereof over the scalp as well as over the hair, while avoiding dripping during application, for example in the eyes.

Finally, it has noticed that this composition conferred good cosmetic properties on the hair, and in particular the shine, the ease of untangling, the smoothing, that the hair was light and became greasy again less quickly, and that it got in shape very easily, and durably.

Thus, according to a first aspect, the invention relates to a hair cosmetic composition in a form suitable for topical application to the hair and the scalp, which comprises a mixture of at least one microbial polysaccharide, at least one modified starch and at least one cationic surfactant, in a cosmetically-acceptable carrier, and which is free of silicones and quaternary ammoniums. In this composition, the total content of modified starch(es) is comprised between 1 and 5% by weight with respect to the total weight of the composition.

In the present description, the expression “at least one” is equivalent to the expression “one or more” and may be substituted by it.

By free of silicones and quaternary ammoniums, it is meant that the composition does not contain such ingredients, or that it includes a negligible, ineffective amount, in particular, for each, lower than 0.1% by weight, preferably lower than 0.01% by weight and more preferably lower than 0.001% by weight, with respect to the total weight of the composition.

In the context of the present invention, by “silicone”, or “polysiloxane”, it is meant, in accordance with the general acceptance, any organosilicon polymer or oligomer with a linear or cyclic, branched or crosslinked structure, with a variable molecular weight, obtained by polymerisation and/or polycondensation of functionalised silanes, and essentially formed by a repetition of main units in which the silicon atoms are bonded together by oxygen atoms (siloxane bond ≡Si—O—Si≡), hydroxyl radicals or hydrocarbon radicals possibly substituted directly bonded at a carbon atom to said silicon atoms.

Preferably, the composition according to the invention is free of siloxanes/organosilicones, and still more generally of silanes.

By quaternary ammonia, it is meant, in a conventional manner per se, compounds derived from ammonia corresponding to the general formula:

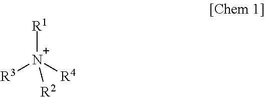

wherein R¹, R², R³ and R⁴ represent alkyl groups with variable length.

In the present description, by “microbial polysaccharide”, it is meant a polysaccharide biosynthesised by a microorganism, for example by a bacterium or a fungus. Such polysaccharides have gelling properties, as well as moisturising properties by a film-forming action, from which the present invention advantageously profits.

The composition according to the invention may contain a single microbial polysaccharide, or a mixture of such polysaccharides.

Preferably, a microbial polysaccharide implemented according to the invention is selected from among microbial gums. Such gums are typically obtained by synthesis of these macromolecules by selected strains of microorganisms under controlled conditions in a fermenter.

A microbial polysaccharide (the microbial polysaccharide when the composition contains only one) implemented according to the invention may be selected from among sclerotium (or scleroglucan) gums such as those produced by the microorganisms of the genus Sclerotium (or Athelia), more particularly of the species Sclerotium rolfsii (also called Athelia rolfsii), gellan gums such as those produced by the microorganisms of the species Pseudomonas elodea (or Sphingomonas elodea), pullulan gums such as those produced by the fungi of the species Aureobasidium pullulans, curdlan gums such as those produced by the microorganisms of the genus Alcaligenes, xanthan gums such as those produced by the microorganisms of the species Xanthomonas campestris, grifolane gums such as those produced by the microorganisms of the species Grifola frondara, lentinane gums such as those produced by the microorganisms of the species Lentinus edodes, Schizophyllan gums such as those produced by the microorganisms of the species Schizophyllum commine, spirulinane gums such as those produced by the microorganisms of the species Spirulina sybsyla and krestin gums such as those produced by the fungi of the species Trametes versicolor.

Preferably, a gum that is particularly interesting in the present invention is the sclerotium gum, produced by the bacteria of the species Sclerotium rolfsii. A particular example of such a microbial polysaccharide is commercially available under the name Amigel®.

In particular embodiments of the invention, the total content of microbial polysaccharide(s), in particular of sclerotium gum, in the composition is comprised between 0.01 and 1% by weight, preferably between 0.1 and 0.5% by weight, and in particular between 0.1 and 0.4% by weight, with respect to the total weight of the composition.

The composition according to the invention may contain a single modified starch, or a mixture of such starches. In the present description, by modified starch, it is meant, in a conventional manner, any derivative obtained by physical, chemical, physicochemical or enzymatic treatment of the starch, for example by baking, substitution, in particular esterification and/or etherification, oxidation, acid hydrolysis, crosslinking, enzymatic conversion, etc.

In particular, the modified starches advantageously have gelling properties, promoting the distribution of the composition over the hair and the scalp, stabilising the composition and increasing rinsability thereof, from which the present invention profits.

According to the invention, the starch may originate from any plant source such as cereals, seeds, tubers, roots, vegetables and fruits.

For example, a starch that can be used in the present invention may be selected from among corn starch, potato starch, pea starch, rice starch, oat starch, barley starch, tapioca starch, wheat starch, cassava starch, and sorgo.

In particular embodiments of the invention, the modified starch is selected from among starch phosphates, obtained for example by esterification of the starch with orthophosphoric acid or a salt thereof, in particular a sodium or potassium salt, by phosphorus oxychloride or by sodium trimetaphosphate, and/or hydroxypropyl starches, which can be obtained for example by etherification of the starch with propylene oxide.

Preferably, a modified starch present in the composition according to the invention is a hydroxypropyl starch phosphate, such as that one commercialised under the name Structure® XL by the company AkzoNobel.

In particular embodiments of the invention, the total content of modified starch(es), in particular of hydroxypropyl starch phosphate, in the composition is comprised between 0.1 and 10% by weight, preferably between 1 and 5% by weight, in particular between 2 and 4.5% by weight, and even more particularly between 3 and 4.5% by weight, with respect to the total weight of the composition.

The composition according to the invention may contain a single cationic surfactant or a mixture of such surfactants, the composition being nonetheless free of quaternary ammoniums.

In the present description, by “cationic surfactant”, it is conventionally meant any surfactant the hydrophilic part of which is positively charged at the pH value of the composition.

In this respect, in preferred embodiments of the invention, the pH of the hair cosmetic composition according to the invention is comprised between 3 and 6, in particular between 3.5 and 5.5 and advantageously between 3.5 and 5, or otherwise between 4 and 6. Such pH values, close to the physiological pH of the healthy scalp, have the effect of improving the shine of the treated hair, by a tightening of its scales.

The adjustment of the pH to the desired value may be done in any conventional manner, in particular by adding in the composition an organic or mineral base, or an inorganic or organic acid, preferably citric acid, lactic acid or hydrochloric acid.

In particular, the cationic surfactant serves as a conditioning agent within the composition according to the invention.

As cationic surfactants, in particular at pH values comprised between 3 and 6, which can be implemented in the composition according to the invention, mention may be made of amidoamines, in particular derived from plants, or the cationic surfactants based on amino acids such as, for example, brassicyl isoleucinate esylate or brassicyl valicate esylate.

In preferred embodiments of the invention, a cationic surfactant implemented in the composition is an amidoamine, in particular selected from among brassicamidopropyl dimethylamine, lauramidopropyl dimethylamine, dimethylamine, stearamidopropyl dimethylamine, myristamidopropyl cocamidopropyl dimethylamine, ricinolamidopropyl dimethylamine, isostearamidopropyl dimethylamine, oleamidopropyl dimethylamine, behenamidopropyl dimethylamine and palmamidopropyl dimethylamine.

Preferably, the hair cosmetic composition according to the invention comprises, as cationic surfactant, at least behenamidopropyl dimethylamine.

In particular embodiments of the invention, the total content of cationic surfactant(s), in particular of behenamidopropyl dimethylamine, in the composition is comprised between 0.1 and 10% by weight, preferably between 0.5 and 8% by weight, in particular between 1 and 5% by weight, more particularly between 1 and 4% by weight, and still more particularly between 2 and 3% by weight, with respect to the total weight of the composition.

By “cosmetically-acceptable carrier”, it is meant, in the present description, that the vehicle is suitable for use by bringing into contact with the keratin materials and the cells of the skin, in particular with the hair and the scalp.

The cosmetically-acceptable carrier used in the hair cosmetic composition according to the invention may comprise water, and/or one or more organic solvent(s), or any mixture thereof.

Preferably, the cosmetically-acceptable carrier is an aqueous carrier. The hair cosmetic composition according to the invention then preferably comprises a water content greater than or equal to 60% by weight, preferably comprised between 60% and 95% by weight, more preferably comprised between 60% and 90% by weight, preferentially comprised between 65% and 90% by weight, in particular comprised between 70 and 90% by weight and for example comprised between 75 and 90% by weight, with respect to the total weight of the composition.

In particularly preferred embodiments of the invention, the composition includes no additional ingredient selected from among fatty substances, or it includes a total amount lower than or equal to 1% by weight with respect to the total weight of the composition, of additional ingredient(s) selected from among fatty substances.

In the context of the present invention, it is meant, by “fatty substance”, an organic compound that is insoluble in water at 25° C., 1 atm, i.e. with a solubility lower than 5% by weight, preferably lower than 1% by weight. In general, the fatty substances are soluble in organic solvents under the same temperature and pressure conditions, such as for example in chloroform, ethanol, benzene or petroleum jelly oil. The expression “fatty substance” encompasses fatty alcohols, fatty acid esters, fatty acids, fatty amides, mineral oils or waxes (or hydrocarbons), animal oils or waxes or plant oils or waxes, etc.

An absence of fatty substances within the hair cosmetic composition according to the invention is particularly advantageous in the context of the application of the composition to the care of both the hair and the scalp, since, on the one hand, after topically applying the composition, the feeling of lightness of the hair is greater, and on the other hand, the effectiveness of care of the scalp is higher, in particular when the cosmetically-acceptable carrier is of the aqueous type, the composition then not being in the form of an emulsion.

In particular embodiments of the invention, the hair cosmetic composition contains a total content of additional ingredient(s) selected from among fatty substances comprised between 0.001% and 1% by weight, in particular comprised between 0.001 and 0.5% by weight, more particularly comprised between 0.001 and 0.2% by weight, and in particular comprised between 0.001 and 0.1% by weight, with respect to the total weight of the composition. Thus, preferably, the hair cosmetic composition according to the invention is not in the form of an emulsion comprising an aqueous phase and a fatty phase. Preferably, the hair cosmetic composition according to the invention is in the form of a more or less thickened liquid, a cream or a gel.

Preferably, the cosmetic composition according to the invention is in the form of a gel, in particular an aqueous gel, allowing in particular advantageously for a better distribution of the composition over the application area and a better impregnation of the keratin materials, in particular the hair, and/or the scalp. Preferably, the hair cosmetic composition according to the invention has a dynamic viscosity, measured at 22° C.±2° C., by means of a rotary viscometer, such as the Mettler Rheomat RM180 viscometer, in particular with a reading time of 10 s, and at a shear rate of 17.7 s-1, which is comprised between 1,200 and 4,000 mPa·s (1,200 and 4,000 centipoise), preferably comprised between 1,500 and 3,500 mPa·s (1,500 and 3,500 centipoise), and still more preferably comprised between 1,500 and 3,000 mPa·s (1,500 and 3,000 centipoises).

The hair cosmetic composition according to the invention can contain any other cosmetically-acceptable ingredient conventional per se in the field of cosmetic care of the scalp and the hair. Preferably, such ingredients are natural and/or of natural origin, derived from renewable resources, in contrast with fossil resources, and they are preferably obtained by environmentally-friendly methods.

Preferably, the hair cosmetic composition according to the invention further contains a small total number of ingredients, in particular a total number of ingredients less than 15, preferably less than or equal to 12.

In preferred embodiments of the invention, the hair cosmetic composition is free of anionic surfactant(s) and/or non-ionic surfactant(s).

According to the present invention, by “anionic surfactant”, it should be understood a surfactant having at least one permanent negative charge or a group that can be ionised into a negatively-charged entity, under the conditions of use of the composition of the invention. By “non-ionic surfactant”, it should be understood a surfactant which does not comprise any charge or any ionisable group.

The hair cosmetic composition according to the invention may further contain a topically active agent for the cosmetic care of the hair and/or the scalp, or a mixture of such active agents.

As such active agents, mention may be made of soothing agents, such as peony extracts, volume agents, such as flax extracts, moisturising agents, such as glycerine, cleansing agents, etc.

The hair cosmetic composition according to the invention may further contain at least one cosmetic additive commonly used in the art, such as, for example, pigments, dyes, mineral fillers, perfumes, proteins, vitamins, natural extracts, diluents, preservatives, pH adjusters, etc. The person skilled in the art will ensure that any selected cosmetic additives and their amount do not affect the properties of the hair cosmetic composition according to the present invention.

Preferably, the hair cosmetic composition according to the invention does not contain a hair dyeing agent.

For example, a hair cosmetic composition according to the invention may contain one or more of the following constituents:—a microbial gum, for example a sclerotium gum, preferably in a content comprised between 0.1 and 0.4% by weight with respect to the total weight of the composition,—a starch phosphate, in particular a hydroxypropyl starch phosphate, preferably in a content comprised between 2 and 4.5% by weight with respect to the total weight of the composition,—an amidoamine, in particular behenamidopropyl dimethylamine, preferably in a content comprised between 1 and 4% by weight with respect to the total weight of the composition,
water, preferably in a content comprised between 65 and 90% by weight with respect to the total weight of the composition,
optionally, an amount comprised between 0.001 and 1% by weight, with respect to the total weight of the composition, of one or more additional ingredient(s) selected from among fatty substances,
optionally, one or more cosmetically-active agent(s),
optionally, one or more cosmetically-acceptable additive(s).

The hair cosmetic composition according to the invention may be used for any application taking advantage of its particularly advantageous properties which include in particular its good ease of distribution over the application area and its good impregnation capacity, which allows obtaining a better action effectiveness over the entire treated area for a smallest possible amount of the composition, and a short laying time, for example in the range of a few seconds to a few minutes, that being so without weighting the hair.

It is particularly suitable for care of keratin materials, in particular the hair, and/or the scalp. Thus, it can consist of a conditioning shampoo, a conditioner, or a care mask for both the scalp and the hair.

Applied to the hair and the scalp, it advantageously facilitates untangling of the hair, improves softness and shine thereof, and it hydrates the latter as well as the scalp. It also features good rinsability.

In particular embodiments of the invention, the hair cosmetic composition is packaged in a delivery device including an elongate-shaped applicator tip. Such a feature advantageously facilitates the application of a controlled amount, and with a good distribution, of the composition over the scalp, line-by-line, as well as over the entire hair.

Another aspect of the invention relates to the cosmetic, non-therapeutic use of a hair cosmetic composition according to the invention, in particular for the care of the hair and/or the scalp. Preferably, this use comprises the topical application of the hair cosmetic composition to the hair and/or the scalp. Preferably, this application is carried out after a shampoo, in the context of a conditioning treatment. Preferably, it is carried out, on the scalp and the lengths of the hair, line-by-line, where appropriate by means of an applicator tip equipping the reservoir in which the composition is contained.

After application, the hair cosmetic composition according to the invention may be rinsed or not. Preferably, the use of the composition according to the invention comprises a step of rinsing the composition, after a possible stand time which may be from a few seconds to a few minutes, preferably from 10 seconds to 5 minutes, preferably from 30 seconds to 5 minutes and still more preferably from 2 to 3 minutes. A massage of the scalp may optionally be carried out during this stand time.

The hair cosmetic composition according to the invention may be applied to the scalp and the hair as often as necessary, in particular every day, concomitantly with or after a shampoo.

According to another aspect, the present invention relates to a method for the non-therapeutic cosmetic care of the hair and/or the scalp, by means of a hair cosmetic composition according to the invention, this method preferably comprising topically applying this hair cosmetic composition according to the invention to the hair and/or the scalp.

This method may meet one or more of the features described hereinbefore with reference to the use of the hair cosmetic composition according to the invention. In particular, it can comprise the application of this composition to the hair and/or the scalp after a shampoo, this application possibly being followed by rinsing, after an optional stand time associated or not with a massage of the scalp.

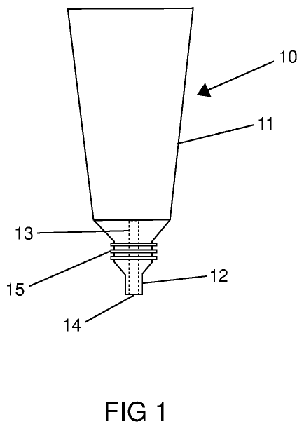

The features and advantages of the invention will appear more clearly in light of the examples of implementation hereinafter, provided as a simple and in no way limiting illustration of the invention, with reference to FIG. 1, which schematically shows a delivery device in which a hair cosmetic composition according to the invention is packaged.

### Example 1—Composition Based on Peony Extract

A hair cosmetic composition in accordance with the invention C1, based on a peony extract as a cosmetic active ingredient, comprises the components indicated in Table 1 hereinafter.

In this table, the components of a commercially available comparative composition Comp1, not in accordance with the invention, are also described.

TABLE 1

| Component | C1 (% w/w) | Comp1 (% w/w) |
|---|---|---|
| Water | 65-90 | 65-90 |
| Glycerine | 1-6 | 1-6 |
| Preservative | 1-5 | 1-5 |
| Sclerotium gum | 0.1-0.4 | 0 |
| Hydroxypropyl starch phosphate | 3-4.5 | 5 |
| Behenamidopropyl dimethylamine | 1-4 | 1-4 |
| Hydrogenated castor oil | 0 | 0.5-2 |
| Lactic acid | 0.1-1.5 | 0.1-1.5 |
| Hydrated betaine | 1-6 | 1-6 |
| Trisodium ethylenediamine disuccinate | 0.1-0.5 | 0.1-0.5 |
| Peony dry extract (Paeonia lactiflora) | 0.07 | 0.07 |
| Perfume | 0.2-1 | 0.2-1 |

The composition C1 in accordance with the invention is in the form of a high-tolerance aqueous gel, which can be used in particular as a conditioner. In particular, when applied topically to the scalp and the hair, it features a soothing effect on the irritation and discomfort feels of the scalp, and that being so as of the first use, this effect being felt durably. It also has a hydrating action on both the scalp and the lengths of the hair. The latter is easier to untangle, softer and shiner.

For example, the composition C1 in accordance with the invention may be used by application on the scalp line-by-line and then over the lengths, then rinsing after 2 to 3 min of stand.

### Example 2—Sensory Profiles of the Compositions C1 and Comp1

This test aims to compare the sensory characteristics of the composition according to the invention C1 with those of the comparative composition Comp1.

The test has been carried out by a hairdresser, trained in the sensory technique for hair products. She evaluates the selected descriptors by the sensory profile method on 12 volunteers coming from a panel for the sensory analysis of topical products, with half-long hairs washed 48 h before the test, who evaluate the sensation of freshness and of soothing.

The volunteers have all healthy hair types.

A shampoo (10 ml) is carried out with a reference formula. After having wrung out the hair, a line is made starting from the top of the skull in order to separate the hair into two half-heads (the hair of the upper portion, attached using a clamp, will not receive any product).

The composition according to the invention C1 or the comparative composition Comp1 are applied using cannula tubes by the hairdresser (2-5 ml), line-by-line on the scalp and then over the lengths of the side imposed by the randomisation, and left to stand for 2-3 minutes. The amount of composition is adapted by the hairdresser according to the hair of the subject. The quotation of the descriptors “phase 1” is carried out.

After rinsing (20 s) of each half-head, the quotation of the descriptors “phase 2” is carried out.

Finally, after drying, the quotation of the descriptors “phase 3” is carried out.

Descriptors of Phase 1 (during application):
Ease of distribution (Distribution 1): ability of the products to be spread over the hair;
Impregnation of the hair (Impregnation 1): impregnation of the product on the hair;
Oily touch (Oily 1): feel like cooking oil between the fingers;
Ease of untangling (Untangling 1): resistance to the passage of the comb into the hair;
Freshness* (Freshness 1): sensation of decrease in the temperature of the scalp;
Heat* (Heat 1): increase in the temperature of the scalp on the application area;
Tension* (Tension 1): rigidity of the scalp when moved;
Stinging* (Stinging 1): presence of “tingling”.

Descriptors of Phase 2 (during/after rinsing):
Ease of rinsing (Rinsing 2): comparison of the speed of removal of the two products;
Ease of untangling (Untangling 2): resistance to the passage of the comb into the hair.

Descriptors of Phase 3 (after drying with fingers):
Electricity (Electricity 3): upon passage of the comb;
Shine (Shine 3): ability of the hair to reflect light;
Softness (Softness 3): silky touch of the hair;
Flexibility (Flexibility 3): loosening up of the loop after drying on a roll;
Volume (Volume 3): swelling of the hair;
Heaviness (Heaviness 3): flattening of the hair.

The descriptors followed by “*” are the descriptors evaluated by volunteers (panel members trained with topical products).

All these descriptors have been selected by the hairdresser trained on the descriptive analysis of care products.

A computer survey presents the 0-100 scales of unstructured analog measurements, for each of the descriptors at the different phases on the computer screen. For each descriptor, the intensities of the 2 compositions are quantified over 2 scales, one corresponding to the product applied to the right, the other to the product applied to the left.

The statistical tests are carried out using the software FIZZ Traitement 2.60 (Biosystèmes).

The analysis is carried out for each descriptor. The significance of the differences between the products is verified by a Student test.

The obtained results, expressed in the form of an average, are indicated in Table 2 hereinafter.

TABLE 2

| Attribute | Composition C1 | Composition Comp1 |
|---|---|---|
| Distribution 1 | 81.1 +/− 3.2 | 68.5 +/− 2.9 |
| Impregnation 1 | 41.4 | 40.6 |
| Oily 1 | 38.2 | 38.2 |
| Untangling 1 | 51.9 | 53.8 |
| Freshness 1 | 54.3 | 54.9 |
| Heat 1 | 0 | 0 |
| Tension 1 | 7.7 | 7.5 |
| Stinging 1 | 2.3 | 0.8 |
| Rinsing 2 | 60.1 | 62.4 |
| Untangling 2 | 66.9 | 68.3 |
| Electricity 3 | 6.4 | 4.8 |
| Shine 3 | 73.3 | 72.5 |
| Softness 3 | 60.8 | 56.1 |
| Flexibility 3 | 64.1 | 68.1 |
| Volume 3 | 74.0 | 75.7 |
| Heaviness 3 | 19.0 | 13.4 |

These results showing all quotations of all descriptors of the two compositions allow highlighting the advantage of the composition according to the invention C1. Indeed, as regards the descriptor “Distribution 1”, the difference between the two products is statistically significantly different. As regards the composition Comp1 (currently on the market), the quotation of “Distribution 1” in percentage is 68.5±2.9, while for the composition according to the invention C1, this quotation reaches 81.1±3.2 (P<0.0001). This better distribution allows for a greater effectiveness of the composition on the scalp, but also over the entire length of the hair.

For all of the other descriptors, the difference between the value obtained for the composition according to the invention C1 and the comparative composition Comp1 is not significant.

Thus, as shown by the other quotations, the increase in the distribution of the composition for the composition according to the invention C1 does not affect the effectiveness of this composition, the untangling, in particular during rinsing of the hair, being in particular very well quoted. Similarly, the criteria evaluated after drying, like shine, softness, flexibility or volume are particularly appreciated. Moreover, the composition according to the invention C1 is perfectly well felt by the volunteers, indeed the notations on sensations like heat, tension and stinging appear close to zero. On the other hand, the freshness effect on the scalp is very marked (score>50).

These advantageous effects of the composition according to the invention C1, in particular the clear improvement of the distribution compared to the comparative composition Comp1, are obtained without significantly affecting the flexibility and the lightness of the hair.

### Example 3—Composition Based on Flax Seed Extract

A hair cosmetic composition in accordance with the invention C2, based on a flax seed extract as a cosmetic active ingredient, comprises the components indicated in Table 3 hereinafter. In this table, the components of a commercially available comparative composition Comp2, not in accordance with the invention, are also described.

TABLE 3

| Component | C2 (% w/w) | Comp2 (% w/w) |
|---|---|---|
| Water | 65-90 | 65-90 |
| Preservative | 1-5 | 1-5 |
| Sclerotium gum | 0.1-0.4 | 0.1-0.4 |
| Hydroxypropyl starch phosphate | 3-4.5 | 0 |
| Behenamidopropyl dimethylamine | 1-4 | 0 |
| Quaternary ammonium | 0 | 1-4 |
| Lactic acid | 0.1-1.5 | 0.1-1.5 |
| Sodium benzoate | 0.2-0.5 | 0.2-0.5 |
| Acetic acid | 0.5-2.5 | 0.5-2.5 |
| Dry flax seed extract | 0.00125 | 0.00125 |
| Perfume | 0.2-1 | 0.2-1 |

The composition C2 in accordance with the invention is in the form of a high-tolerance aqueous gel, which can be used in particular as a conditioner. For example, this composition may be used by application on the scalp line-by-line and then over the lengths, then rinsing after 2 to 3 min of stand. Advantageously, it allows well controlling the volume of the hair.

### Example 4—Sensory Profiles of the Compositions C2 and Comp2

The sensory characteristics of the composition according to the invention C2 and of the comparative composition Comp2 are established as described in Example 2 hereinabove, with only some of the descriptors.

The obtained results, expressed in the form of an average, are indicated in Table 4 hereinafter, for the attributes for which statistically significant differences (with an alpha threshold of 5%) have been demonstrated.

TABLE 4

| Attribute | Composition C2 | Composition Comp2 | Difference |
|---|---|---|---|
| Distribution 1 | 73.83 +/− 17.63 | 59.42 +/− 15.01 | 14.42 |
| Impregnation 1 | 66.42 +/− 12.03 | 58.25 +/− 8.99 | 9.28 |
| Untangling 2 | 73.58 +/− 11.39 | 59.08 +/− 13.95 | 9.27 |
| Electricity 3 | 5.17 +/− 7.76 | 21.58 +/− 17.05 | −16.42 |

These results clearly prove the superiority of the composition according to the invention C2 in terms of distribution and impregnation, two major criteria pursued in particular for conditioners. This improvement in the distribution for the composition according to the invention is not made to the detriment of the lightness and flexibility of the hair, which are similar for both tested compositions.

These results also show that the composition according to the invention C2 allows an easier untangling during rinsing. Besides the descriptors of interest, it is also less oily, and it renders the hair less electrical, if not electrically at all, compared to the composition Comp2 currently on the market. Moreover, the composition C2 according to the invention confers, after drying the hair, a volume, a shine and a softness that are quite remarkable.

### Example 5—Packaging

The hair cosmetic composition according to the invention may be packaged in a delivery device as schematically shown in FIG. 1.

This device 10 includes a main body 11 forming a reservoir for the hair cosmetic composition. This main body is equipped with an applicator tip 12, with an elongate shape according to the longitudinal axis of the body 11. This applicator tip 12 is pierced with a through channel 13, terminating in a first end by a delivery opening 14 and opening at an opposite end into the body 11, so as to enable the circulation of the hair cosmetic composition which is contained in the latter up to the delivery opening 14.

Preferably, the applicator tip 12 is provided on its external periphery with a thread 15 intended for the removable fastening, around the applicator tip, of a cap provided with a cooperating thread.

Such a packaging and delivery device of the type having an applicator tip proves to be particularly useful for the application of the hair cosmetic composition according to the invention, in particular as a conditioner, on the scalp and over the length of the hair.

What is claimed is:

1.-18. (canceled)

19. A hair cosmetic composition suitable for topical application, comprising a mixture of at least one microbial polysaccharide, at least one modified starch and at least one cationic surfactant, in a cosmetically-acceptable carrier, and free of silicones and quaternary ammoniums, wherein the total content of modified starch(es) in the composition is comprised between 1 and 5% by weight with respect to the total weight of the composition.

20. The hair cosmetic composition according to claim 19, wherein said cosmetically-acceptable carrier is an aqueous carrier and said composition contains at least 60% by weight of water, with respect to the total weight of the composition.

21. The hair cosmetic composition according to claim 19, including no additional ingredient selected in the group consisting of fatty substances.

22. The hair cosmetic composition according to claim 19, having a dynamic viscosity, measured at 22° C. by rotary viscometer at a shear rate of 17.7 s-1, comprised between 1,200 and 4,000 Pa·s.

23. The hair cosmetic composition according to claim 19, free of anionic surfactants and/or non-ionic surfactants.

24. The hair cosmetic composition according to claim 19, further containing an active agent for cosmetic care of the hair and/or the scalp.

25. The hair cosmetic composition according to claim 19, further containing at least one moisturising agent.

26. The hair cosmetic composition according to claim 25, further containing glycerine as a moisturising agent.

27. The hair cosmetic composition according to claim 19, wherein the content of modified starch(es) is comprised between 2 and 4.5% by weight with respect to the total weight of the composition.

28. The hair cosmetic composition according to claim 19, wherein the content of microbial polysaccharide(s) is comprised between 0.1 and 0.5% by weight with respect to the total weight of the composition.

29. The hair cosmetic composition according to claim 28, wherein the content of microbial polysaccharide(s) is comprised between 0.1 and 0.4% by weight with respect to the total weight of the composition.

30. The hair cosmetic composition according to claim 19, wherein the content of cationic surfactant(s) is comprised between 1 and 4% by weight with respect to the total weight of the composition.

31. The hair cosmetic composition according to claim 19, wherein one microbial polysaccharide is a sclerotium gum.

32. The hair cosmetic composition according to claim 19, wherein one modified starch is hydroxypropyl starch phosphate.

33. The hair cosmetic composition according to claim 19, wherein one cationic surfactant is behenamidopropyl dimethylamine.

34. The hair cosmetic composition according to claim 19, free of silanes.

35. The hair cosmetic composition according to claim 19, including a total amount lower than or equal to 1% by weight with respect to the total weight of the composition, of additional ingredient(s) selected in the group consisting of fatty substances.

36. A method for the non-therapeutic cosmetic care of the hair and/or the scalp, comprising a step of topically applying to the hair and/or the scalp a hair cosmetic composition according to claim 19.

37. The method according to claim 36, wherein said hair cosmetic composition is applied to the hair and/or the scalp after a shampoo.
